(19) United States
(12) Patent Application Publication
(10) Pub. No.: US 2003/0225427 A1
(43) Pub. Date: Dec. 4, 2003

(54) Wristband type electric nose cleaner

(76) Inventor: To-Chan Chen, Taipei (TW)

Correspondence Address: Hsu & Associates, P.O. Box 72, Nankang, Taipei, 115, TW

(21) Appl. No.: 10/164,507

(22) Filed: Jun. 3, 2002

(51) Int. Cl. A61F 9/00

(52) U.S. Cl. 606/162

(57) ABSTRACT

A wristband type electric nose cleaner provides an electric spray-suction pump assembly, which applies the Bernoulli's theorem to provide a function of atomization for the cleaning solution. A spray device and a suction device are involved in the nose cleaner such that the foreign matters such as mucus can be sucked out of the nose and the cleaning solution such as normal saline can be injected into the deep inside nasal cavity in a state of being atomized to enhance the nose clean job. Furthermore, a wristband is attached to the nose cleaner for fixing the nose cleaner firmly to the wrist of a user to prevent the nose cleaner from damage resulting from falling from the hand.

### BACKGROUND OF THE INVENTION

1. Field of the Invention

The present invention relates to a wristband type nose cleaner, and, particularly, to a portable house electric nose cleaner, which performs washing the nose with mist spray or removing foreign matters in the nose with sucking air.

2. Description of Related Art

The nose cleaner available in the market such as a push type nose washer, a push type nose suction device or a mouth aspirating type nose suction device has a single function only and has to be operated manually. The conventional nose cleaners usually provide an extensible container for being pressed and released repeatedly with the hand of a user or being sucked with the mouth of the user such that the air in the container can be pressed to constitute fast moving air for cleaning the nose by way of being sprayed or sucked. Although the conventional nose cleaners are not bad from the standpoints of the principle applied and the way of design, problems such as unsuitable for use and improper regulated air pressure are still there. A further detail with regard to the disadvantages is stated hereinafter.

During the manual push type nose washer or the manual push type suction device being operated manually by the user in a way of pressing and releasing the extensible container repeatedly, a defect worth us to care is it is hard to control the pressing force appropriately. Especially for the infant, the mucous membrane in the infant's nose is very weak and it is very easy to become hurt if the cleaning solution (such as normal saline) is sprayed with an excessively large force or the foreign matter (such as nasal mucus) is sucked out of the nose with an excessively large force. If the force is excessively small, the cleaning solution is unable to eject smoothly or the foreign matter is not possible to be sucked out completely. By the same token, the conventional mouth aspirating type suction device also has the problem of pressing force with a possibility of indirect infection of cold virus (such as droplet infection through mouth aspirating).

During the push type nose washer or the push type suction device being used, the repeated movements of pressing and releasing cause the extensible container being in a state of shaking and it is hard to locate the contact port thereof at the nose. In this way, it is not convenient to be operated and it is very likely to hurt the mucous membrane in the nose.

The cleaning solution ejected from the push type nose washer is a liquid state to forcedly wash the nasal cavity so that it is easy to irritate the nose or to cause cough if the cleaning solution stimulates the mucous membrane of the nasal cavity too much. Further, the cleaning solution ejected by the push type nose washer is not possible to become atomized so that it is hard to clean the deeper inside of the nasal cavity and a great deal of cleaning solution has to be used.

It is not easy to carry or store up the push type suction device and the mouth aspiration suction device and the container thereof for receiving the foreign matter such as nasal mucus sucked out of the nose is made of material not resisting the high temperature of boiling water during the process of boiling sterilization.

Further, The preceding conventional nose cleaners provide undesirably effect of nose cleaning.

The conventional nose cleaners available in the market are usually designed to have a hand grip respectively so that it is easy to fall down carelessly and become damaged.

As the foregoing, it is known that there are disadvantages while the conventional nose cleaners are in use. Moreover, when the conventional nose cleaners are used for cleaning the infant's nose cavity, it has to be taken extreme care during performing the nose clean job.

### SUMMARY OF THE INVENTION

The crux of a wristband type electric nose cleaner according to the present invention resides in that the wristband type electric nose cleaner includes a casing, a pump being fixed in the casing, a motor with an eccentric shaft being joined to rear side of the pump, a suction device detachably engaged to an end of the pump and a sprayer can detachably engaged to the end of the pump. When the motor is started to run, foreign matters can be sucked out of the nose in case of the suction device being mounted to the pump and clean solution can be sprayed deeply into the nose in case of the sprayer being mounted to pump instead of the suction head.

Accordingly, a primary object of the present invention is to enhance the nose clean job so that the nose cavity can be cleaned more effectively and conveniently with reduced cleaning solution needed.

Another object of the present invention is to promote the effectiveness of the cleaning solution because of the cleaning solution being atomized such that it is helpful for the atomized cleaning solution to reach deeper inside the nasal cavity and to avoid occurring nose irritating or throat coughing.

A further object of the present invention is to avoid mucous membrane of the nasal cavity being stimulated or hurt during the nose clean job.

A further object of the present invention to provide an electric nose cleaner, which has a compact size for being portable conveniently, with a wristband can be attached to a wrist firmly to avoid damage resulting from falling down from the hand.

A further object of the present invention to provide a wristband type electric nose cleaner, which can spray atomized cleaning solution into the nose and can suck the foreign matters out of the nose instead with facility regardless the power source is alternate current or direct current.

### BRIEF DESCRIPTION OF THE DRAWINGS

The present invention can be more fully understood by reference to the following description and accompanying drawings, in which:

FIG. 1 is an exploded perspective view of a pump in a wrist band type nose cleaner according to the present invention;

FIG. 2 is an exploded perspective view of a suction device in the wristband type electric nose cleaner of the present invention;

FIG. 3 is a sectional view illustrating the pump shown in FIG. 1 and the suction device shown in FIG. 2 having been associated with each other for performing the suction function;

FIG. 4 is an exploded perspective view of a sprayer in the wristband type electric nose cleaner of the present invention;

FIG. 5 is an enlarged perspective view of a part enclosed by a circle shown in FIG. 4;

FIG. 6 is a sectional view illustrating the sprayer shown in FIG. 4 having been joined to the pump shown in FIG. 1 for performing the spraying function;

FIG. 7 is a sectional view similar to FIG. 5 illustrating another arrangement of the pump for performing the spray function;

FIG. 8 is a perspective view of a connective seat shown in FIG. 7; and

FIG. 9 is a perspective view of the wristband type nose cleaner of the present invention illustrating an appearance thereof.

### DETAILED DESCRIPTION OF THE PREFERRED EMBODIMENTS

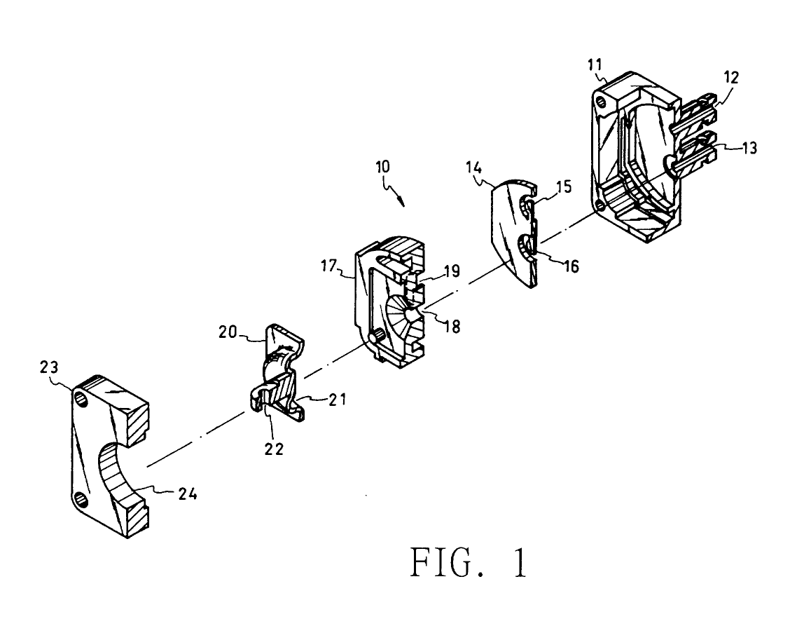

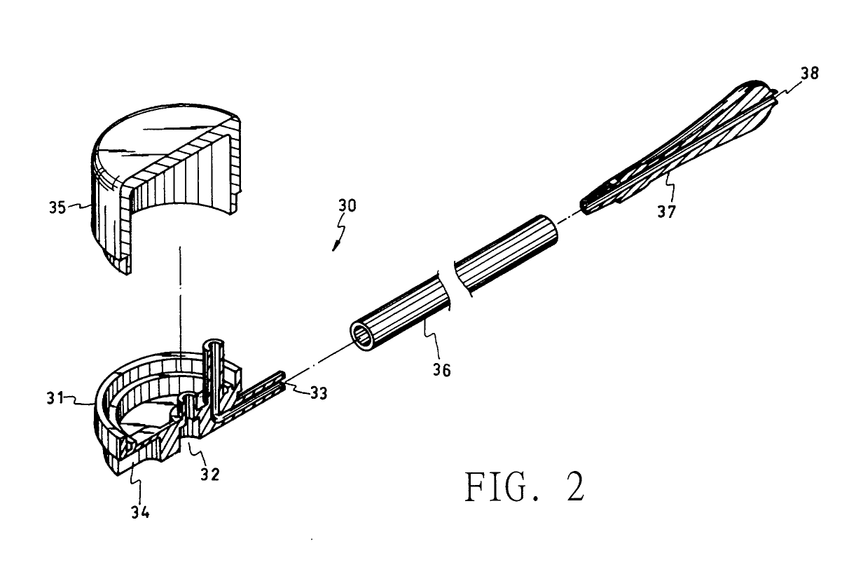

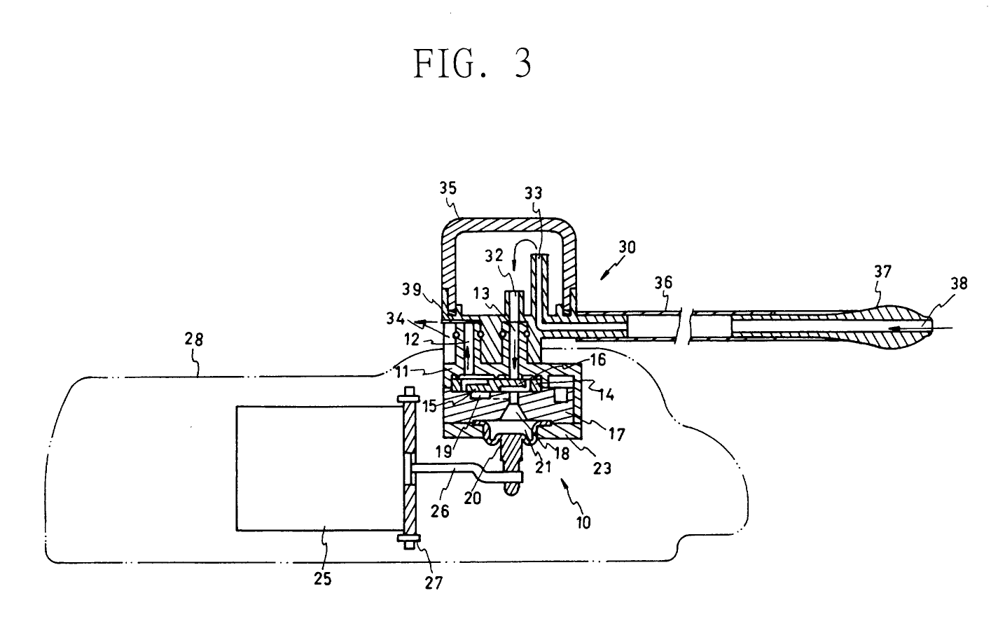

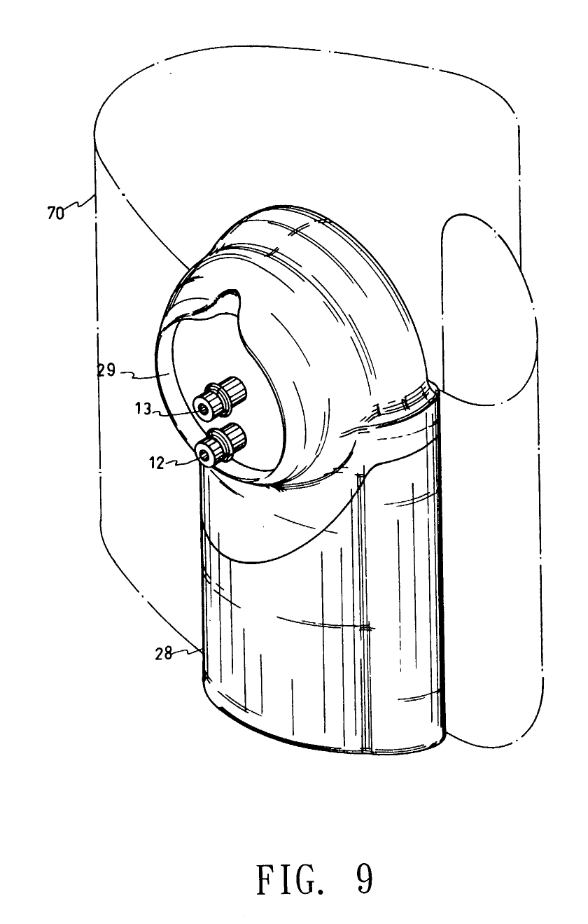

Referring to FIGS. 1, 2 and 3, a wristband type electric nose cleaner of the present invention has a pump 10 and a suction device 30, wherein, the pump 10 is fixedly attached to the inner side of a casing 28. The pump 10 has a pump body 11 with a spray outlet tube 12 and a suction inlet tube 13 extending outward the casing 28. A spray-suction valve plate 14 is arranged in the pump body 11 and a valve cover 17 fits with the pump body 11 at a lateral side of the pump body 11 next to the valve plate 14. The valve plate 14 has a spray valve gate 15 and a suction valve gate 16 and the valve cover 17 has an air spray chamber 19 and an air hole 18. The air hole 18 is arranged to communicate with the air spray chamber 19, and the air hole 18 can communicate with or disconnect from the suction inlet tube 13 by way of controlling the suction valve gate 16. Similarly, the air spray chamber 19 can communicate with or disconnect from the spray outlet tube 12 by way of controlling the spray valve gate 15. A sucking disk 20 is disposed at a lateral side of the valve cover 17 opposite to the valve plate 14 and is fixedly attached to the valve cover 17 with a pump cap 23. The sucking disk 20 has an air chamber 21 corresponding to the air hole 18 and an axial projection stem with a shaft hole 22 to pass through an axial bore 24 in the pump cap 23. The shaft hole 22 is passed through with an eccentric shaft 26 on a motor 25 such that the axial projection stem of the sucking disk 20 can reciprocate to result in the air chamber 21 being compressed and released repeatedly during the motor 25 is in a state of running. In this way, the suction gate 16 of the valve plate 14 can open and shut the suction inlet tube 13 and the suction inlet tube 13 can detach from the suction gate 16 to admit the air current outside the suction inlet tube 13 while the air chamber 21 is released and the suction inlet tube 13 is shut with the suction gate 16 and the air in the air chamber 21 passes through the spray chamber 19 from the air hole 18 to open the spray valve gate 15 and discharges via the spray outlet tube 12. The suction device 30 has a suction cover 31 with a sucking hole 32 is inserted into a joint fixing base 29 on the casing 28 as shown in FIG. 9 so that the sucking hole 32 can align with the suction inlet tube 13. The suction cover 31 at the lateral side thereof has an engaging recess part 34 to join the spray outlet tube 12. The engaging recess part 34 has a discharge recess 39 being disposed to fit and communicate with the spray outlet tube 12 so that the air can be discharged via the discharge recess 39 and the spray outlet tube 12. A suction tube 33 is provided on the spray cover 30 next to a lower end of the suction hole 32 to connect with a suction tube 37 via a connecting tube 36. The suction tube 37 with a sucking passage 38 sucks the foreign matter in the nasal cavity into the nose cleaner. Further, a suction container 35 fits with the suction cover 31 to receive the foreign matter.

As the foregoing, once the motor 25 is started to run, the eccentric shaft 26 actuates the air chamber 21 of the sucking disk 20 in the pump 10 to perform the repeated movements of compressing and releasing such that the spray valve gate 15 and the suction valve gate 16 can react a reverse movement respectively with respect to the spray chamber 19 and the suction inlet tube 13 to result in one of the gates 15, 16 being opened and the other one of gates 15, 16 being shut alternately. The suction inlet tube 13 attracts the air in the suction container 35 to enter the air hole 18 via the sucking hole 32 at the moment of the air chamber 21 stretches inversely toward the air hole 18. The suction valve gate 16 can be pushed toward the suction inlet tube 13 to close the suction inlet tube 13 at the moment of the air chamber 21 being pushed by the eccentric shaft 26 and moving toward the air hole 18. In the meantime, the air from the air hole 18 pushes the spray gate 15 away via the spray chamber 19 and the air can be discharged from the spray outlet tube 12 via the discharge recess 39 such that the suction container 35 becomes in a state of vacuum and the foreign matter in the nose can be sucked into the suction container 35. Therefore, the spray valve gate 15 and the suction valve gate 16 opening and shutting alternately allows the suction container 35 being in a state of semi-vacuum or full of the air so that the foreign matter can stay in the suction container 35 without entering the sucking hole 32.

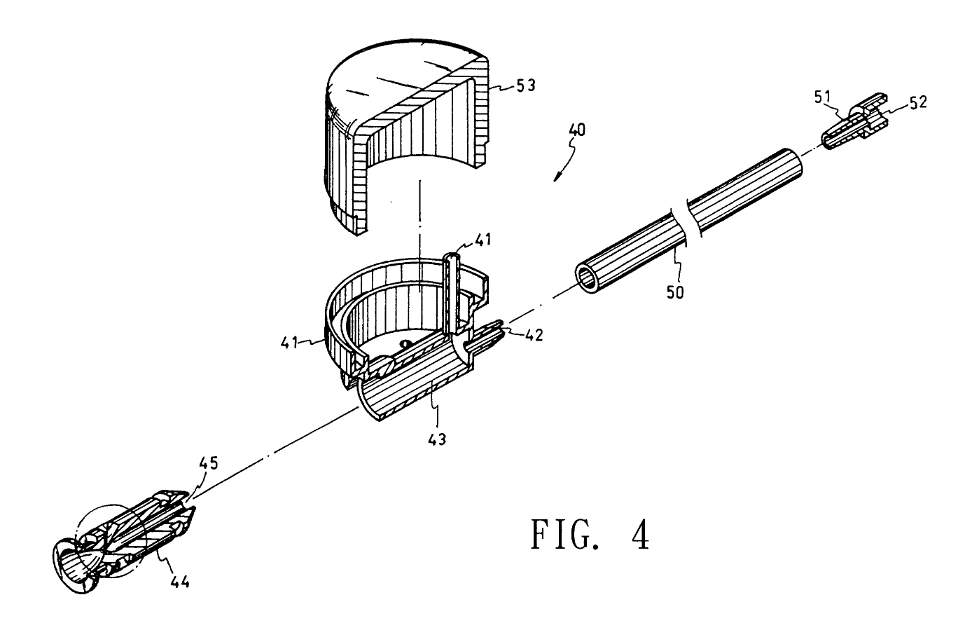

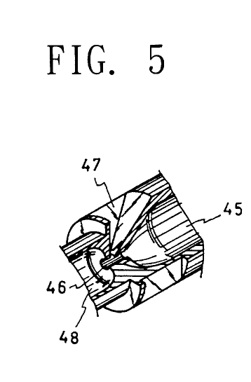

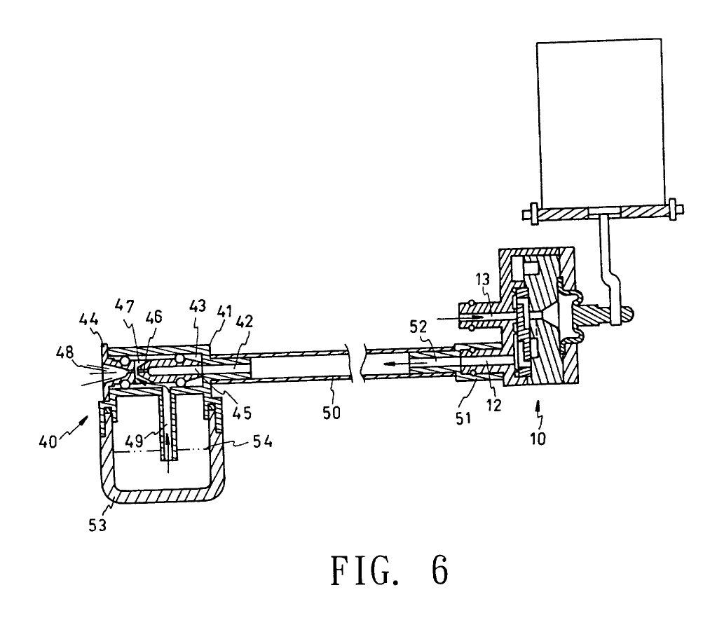

Referring to FIGS. 4, 5 and 6, the sprayer 40 has a spray cover 40 with an atomization chamber 43 and a spray nozzle 44 is arranged in the atomization chamber 43 such that an air passage 45 in the nozzle 44 communicates with an air adapter 42 extending from the atomization chamber 43. The air passage 45 at another end thereof extends an atomization hole 46 to communicate with a liquid groove 47 at the lateral side thereof and at an end of the atomization hole 46 is provided with a spray hole 48. The liquid groove 47 further communicates with a liquid tube 49 attached to the atomization chamber 37 on the sprayer cap 41. The air adapter 42 fits with an end of a connecting tube 50 and a connector 51 at an end thereof fits with another end of the connecting tube 50. Once another end of the connector 51 is connected to the spray outlet tube 12 of the pump 10, the air is sucked into the pump 10 via the suction inlet tube 13 as soon as the motor 25 is started to run. Then, the air moves along an air passage 52 in the connector 51 and the connecting tube 50 to enter the air passage 45 via the adapter 42 so that the air can be ejected via the atomization hole 46 and the spray hole 48. When the air passes through the atomization hole 46 and the liquid groove 47 becomes in a state of vacuum, the cleaning solution 54 in the spray container 53 is sucked to the atomization hole 46 via the liquid tube 49 to mix with the air and the mixture can be atomized before being injected into the nose from the spray hole 48 for cleaning the nasal cavity.

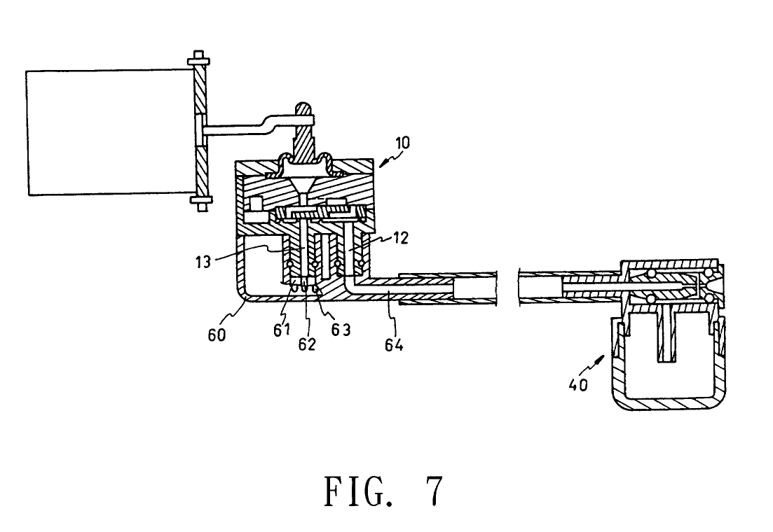

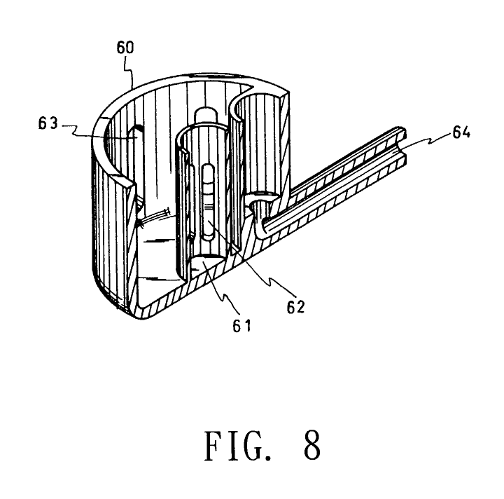

Referring to FIGS. 7 and 8, another embodiment of the present invention is illustrated. It can be seen that a connecting seat 60, which is fixedly attached to the pump 10, is provided with a plurality of air grooves 63 at the inner wall thereof. An airflow tube 61 with air grooves 62 arranged at the tube wall thereof is provided in the connecting seat 60 and the airflow tube 61 is fixedly attached to the suction inlet tube 13. Hence, the air can enter the airflow tube 61 and the suction inlet tube 13 after coming into the connecting seat 60 through the air grooves 63 and moves toward the sprayer 40 via the spray outlet tube 12. Further, an air guide tube 64 extends from the connecting seat 60. The connecting seat 60 can be used instead of the preceding connector 51 such that it can prevent the user from connecting the connector 51 to the suction inlet tube 13 carelessly.

Referring to FIG. 9, it can be seen from the appearance of the electric nose cleaner of the present invention that the spray outlet tube 12 and the suction inlet tube 13 jut out from the joint fixing base 29 of the casing 28 thereof in a way of being disposed at asymmetrical positions to each other with respect to the center of the joint fixing base 29. In this way, the mistake resulting from the spray outlet tube 12 and the suction inlet tube 13 are misconnected during the suction device 30 and the connecting seat 60 being inserted into the joint fixing base 29. Further, the casing 28 at the rear side thereof is attached with a wristband 70 such that the electric nose cleaner of the present invention can be located at a wrist of the user and the nose cleaning job for the nose patient can be performed with facility. Moreover, the power source and a control circuit for the power switch can be arranged in the nose cleaner of the present invention to ease the operation of the nose cleaning.

While the invention has been described with reference to the preferred embodiments thereof, it is to be understood that modifications or variations may be easily made without departing from the spirit of the invention, which is defined by the appended claims.

What is claimed is:

1. A wristband type electric nose cleaner, comprising:
a casing with a joint fixing base;
a pump with a front side and a rear side, being fixed in the casing;
a motor with an eccentric shaft, being rotatably joined to the rear side of the pump;
a suction device, being detachably engaged to the front end of the pump; and
a sprayer, being detachably engaged to the front end of the pump;
whereby, when the motor is started to run, foreign matters can be sucked out of the nose in case of the suction device being mounted to the pump and clean solution can be sprayed into the nose in case of the sprayer being mounted to pump instead of the suction head.

2. The wristband type electric nose cleaner according to claim 1, wherein the pump further comprises
a pump body with a front side and a rear side, extending a spray outlet tube and a suction inlet tube from the front side thereof;
a valve cover, being received in and fitting with the pump body, providing an air spray chamber and an air hole with the air spray chamber communicating with the air hole;
a spray-suction valve plate, being arranged between the pump body and the valve cover, having a spray valve gate and a suction valve gate corresponding to the air hole and the air spray chamber respectively;
a sucking disk, being disposed at a lateral side of the valve cover opposite to the valve plate, having an air chamber corresponding to the air hole and an axial projection stem with a shaft hole extending rearward;
a pump cap with an axial bore, being disposed at a lateral side of the sucking disk and being fixedly attached to the pump body such that the projection stem can pass through the axial bore;
wherein, the air hole can communicate or disconnect from the suction inlet tube by way of controlling the suction valve gate and the air spray chamber can communicate or disconnect from the spray outlet tube by way of controlling the spray valve gate; and the shaft hole in the axial projection stem can be inserted with the eccentric shaft of the motor;
whereby, the sucking disk is actuated to allow the air chamber reciprocating and the air can be compressed to move from the suction inlet tube to the spray outlet tube for being discharged via the air hole and the air spray chamber.

3. The wristband type electric nose cleaner according to claim 1, wherein the suction device further comprises
a suction cover, having two sucking holes, one of the sucking holes being provided to align with the suction inlet tube, providing an engaging recess part to join the spray outlet tube, the engaging recess part having a discharge recess being disposed to fit and communicate with the spray outlet tube;
a first connecting tube with two ends, one of the ends being fitted to the other one of the sucking holes; and
a suction tube, having a rear end thereof fitting with the other end of the first connecting tube, having an axial sucking passage communicating with the first connecting tube; and
a suction container, being fixed to the suction cover;
whereby, the air can be discharged via the discharge recess and the spray outlet tube and the suction tube can suck the foreign matter in the nasal cavity of a user into the suction container.

4. The wristband type electric nose cleaner according to claim 1, wherein the sprayer further comprises
a spray cover, providing an atomization chamber at a side thereof with an air adapter axially extending from the atomization chamber and a liquid tube extending vertically from the atomization chamber;
a solution container, being fixedly attached to the spray cover at another side thereof;
a spray nozzle, being arranged in the atomization chamber, providing an airflow hole to communicate with the air adapter at an end thereof and another end of the air passage extending an atomization hole to communicate with a liquid groove at a periphery thereof and at another end of the atomization hole having a spray hole, the liquid groove further communicating with the liquid tube;
a second connecting tube with two ends, one of the ends fitting with the air adapter; and
a connector, connecting with another end of the connecting tube and having an air passage therein.
whereby, once the adapter is connected to the spray outlet tube on the pump, the air is sucked into the pump via the suction inlet tube as soon as the motor is started to run; the air then can pass through the connector and the connecting pipe to enter the air adapter and the airflow hole so that the air can be ejected via the atomization hole and the spray hole; when the air passes through the atomization hole and the liquid groove becomes in a state of vacuum, the cleaning solution in the solution container is sucked to the atomization hole via the liquid tube to mix with the air and the mixture can be atomized and injected into the nose from the spray hole for cleaning the nose.

5. The wristband type electric nose cleaner according to claim 4, wherein the sprayer communicates with the pump by way of a connecting seat.

6. The wristband type electric nose cleaner according to claim 5, wherein the connecting seat has a plurality of air grooves disposed at an inner wall thereof and provides two airflow tubes, one of the airflow tubes having a plurality of air grooves disposed at an inner wall thereof to connect with the suction inlet tube and the other airflow tube is used for communicating with spray outlet tube of the pump and the connecting tube of the sprayer.

7. The wristband type electric nose cleaner according to claim 1, wherein a wristband is attached to the casing.
